(19) United States
(12) Patent Application Publication
(10) Pub. No.: US 2021/0085942 A1
(43) Pub. Date: Mar. 25, 2021

(54) LIQUID APPLICATOR

(71) Applicant: 3M INNOVATIVE PROPERTIES COMPANY, Saint Paul, MN (US)

(72) Inventors: ALAN R. DOMBROWSKI, WOODBURY, MN (US); ROBERT A. ASMUS, HUDSON, WI (US)

(73) Assignee: 3M INNOVATIVE PROPERTIES COMPANY, Saint Paul, MN (US)

(21) Appl. No.: 17/031,964

(22) Filed: Sep. 25, 2020

Related U.S. Application Data
(60) Provisional application No. 62/905,730, filed on Sep. 25, 2019.

(51) Int. Cl. A61M 35/00; A61J 1/06

(52) U.S. Cl. CPC A61M 35/003; A61J 1/067

(57) ABSTRACT

The disclosed applicator includes an activation region having one or more pinch point levers in the cover of the applicator. Within the cover of the applicator is a deformable body and disposed within the deformable body is a frangible ampoule containing a solution. A user activates the pinch point levers to deform the deformable body and crush the frangible ampoule, releasing the solution. The hinging connection of the pinch point lever reduces the break strength needed in order to crush the ampoule, while the deformable body provides a safe barrier to protect the user against glass shards.

### CROSS REFERENCE TO RELATED APPLICATION

This application claims priority to U.S. Provisional Application Ser. No. 62/905,730, filed Sep. 25, 2019, the disclosure of which is incorporated by reference in its entirety herein.

### BACKGROUND

The present disclosure relates to an applicator for applying a liquid to a surface. In particular, the present disclosure relates to an applicator including a frangible ampoule.

Barrier products are used to protect the skin of patients who suffer from a variety of conditions including, for example, urinary and fecal incontinence, skin occlusion, ileostomy and colostomy. The presence of high moisture and corrosive enzymes from intestinal fluids can lead to devastating breakdown of the skin leading to fungal infections and denuding and erosion of the skin. Some of the products used to protect the skin from these challenges include occlusive barrier pastes or liquid film-forming solutions. Barrier pastes are messy to apply as well as clean up and may interfere with securing ostomy devices.

Liquid barrier solutions can be held in an applicator. Certain compositions used in a liquid barrier solution, such as cyanoacrylate, are highly reactive and so are typically contained in glass ampoules. These ampoules are often contained in a liquid applicator where the user breaks the ampoule.

### SUMMARY

Applicators may be used to apply liquid solutions to a surface, such as skin or other tissues. Medical preparations such as, for example, skin barrier products, antiseptics, adhesion enhancing products, adhesive tape trauma protectants, and pharmaceutical agents such as analgesics may be applied as liquid solutions. These solutions may be applied with saturated sponges that are attached to an applicator. Some applicators hold the solution to be applied in a frangible ampoule that requires additional elements to crush the ampoule and release the liquid. However, these applicators are often difficult or cumbersome to use because the applicator wall must be thick in order to protect the user or patient from broken glass shards from the crushed ampoule. The thick wall is challenging to deform and ultimately crush the ampoule contained in the applicator.

The present disclosure provides an applicator that includes an activation region having one or more pinch point levers in the cover of the applicator. Within the cover of the applicator is a deformable body and disposed within the deformable body is a frangible ampoule containing a solution. A user activates the pinch point levers to deform the deformable body and crush the frangible ampoule, releasing the solution. The hinging connection of the pinch point lever reduces the break strength needed in order to crush the ampoule, while the deformable body provides a safe barrier to protect the user against glass shards.

In one embodiment, an applicator for applying a solution to a surface comprises a cover comprising an exterior surface and an activation region, a deformable body disposed within the cover at the activation region, a frangible ampoule disposed within the deformable body, wherein the frangible ampoule contains the solution, a pinch point lever disposed within the activation region of the cover, wherein the pinch point lever is flexibly connected to the exterior surface of the cover. The actuation of the pinch point lever against the deformable body breaks the frangible ampoule.

In one embodiment, the cover further comprises a cover interior surface surrounding a cover cavity. In one embodiment, the cover comprises a distal portion and a proximal portion, wherein the distal portion comprises an opening for releasing the solution from the applicator.

In one embodiment, the deformable body is adjacent to the interior surface of the cover. In one embodiment, the deformable body has a proximal end and a distal end and a midsection, wherein the deformable body has a continuous wall extending circumferentially around the midsection. In one embodiment, the deformable body has an opening at the distal end. In one embodiment, the deformable body comprises a support region including one or more cross walls that extend longitudinally across the support region. In one embodiment, the deformable body comprises one or more raised ridges extending from the interior surface of the deformable body to provide air space between the deformable by and cover. In one embodiment, the deformable body further comprises a flex region, an interior surface and one or more ridges extending along the interior surface at the flex region.

In one embodiment, the pinch point lever includes a hinge portion that is integral to the cover. In one embodiment, the pinch point lever has a free section and a hinge end, wherein the free section is an end of the pinch point lever opposite the hinge end. In one embodiment, the hinge end is connected to the exterior surface of the cover and the free end is disconnected from the exterior surface of the cover. In one embodiment, the pinch point lever has a first hinge end and a second hinge end and a free section between the first hinge end and the second hinge end. In one embodiment, the applicator further comprises a second pinch point lever disposed within the activation region of the cover, wherein the second pinch point lever is flexibly connected to the exterior surface of the cover.

In one embodiment, the applicator further comprises a head region located at the distal portion of the cover, wherein the head region comprises an aperture. In one embodiment, the applicator further comprises absorbent element at the distal portion of the cover. In one embodiment, the ampoule is hermetically sealed.

In one embodiment, a method of applying a solution to a surface comprises providing the applicator, pressing the pinch point lever against the deformable body, breaking the frangible ampoule such that the solution releases through the deformable body and the cover.

### BRIEF DESCRIPTION OF DRAWINGS

FIG. 1 is a side perspective view of an applicator with a pinch point lever;

FIG. 2 is a rear perspective view of the applicator of FIG. 1;

FIG. 3 is an exploded view of the applicator of FIG. 1 including a deformable body and frangible ampoule;

FIG. 4 is a cross-sectional view of the applicator of FIG. 1;

FIG. 5 is a perspective view of the deformable body;

FIG. 6 is a cross-sectional view of the deformable body;

FIG. 7 is a cross sectional view of a cover of the applicator;

FIG. 8 is a side perspective view of a second embodiment of an applicator with a pinch point lever.

### DETAILED DESCRIPTION

### Definitions

The term “a”, “an”, and “the” are used interchangeably with “at least one” to mean one or more of the elements being described.

The term “and/or” means either or both. For example, “A and/or B” means only A, only B, or both A and B.

The terms “including,” “comprising,” and “having,” and variations thereof, are meant to encompass the items listed thereafter, and equivalents thereof, as well as additional items.

The terms “polymer” and “polymeric material” refer to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The terms “copolymer” and “copolymeric material” refer to a polymeric material prepared from at least two monomers.

The terms “longitudinal” and “axial” are used to refer to a direction or axis that is generally parallel to a central longitudinal axis of an applicator and generally parallel to the overall direction of solution flow.

The terms “lateral” and “transverse” are used to refer to a direction or axis that is perpendicular to the central longitudinal axis the longitudinal direction.

The terms “vertical” and “normal” are used to refer to a direction or axis that is normal to both the longitudinal and lateral directions (or axes).

The term “proximal” and “distal” are used to represent longitudinal or axial directions, relative to a user (such as, for example, a medical practitioner) using or holding the applicator. That is, the term “distal” is used to refer to the direction away from the medical practitioner (and toward a surface (such as, for example, a skin surface) to be treated, i.e., to which the applicator will apply a solution); and the term “proximal” is used to refer to the direction toward the user (and away from the surface to be treated). For example, the distal end of an applicator is directed toward, or even pressed against, the surface to be treated, while the proximal end extends away from the surface and toward the user. Similarly, the distal end of any portion or component of an applicator is directed or oriented toward the surface to be treated and is oriented toward the distal end of the applicator or forms or defines the distal end of the applicator. In addition, the proximal end of any portion or component of an applicator is directed or oriented away from the surface to be treated and is oriented toward the proximal end of the applicator or forms or defines the proximal end of the applicator.

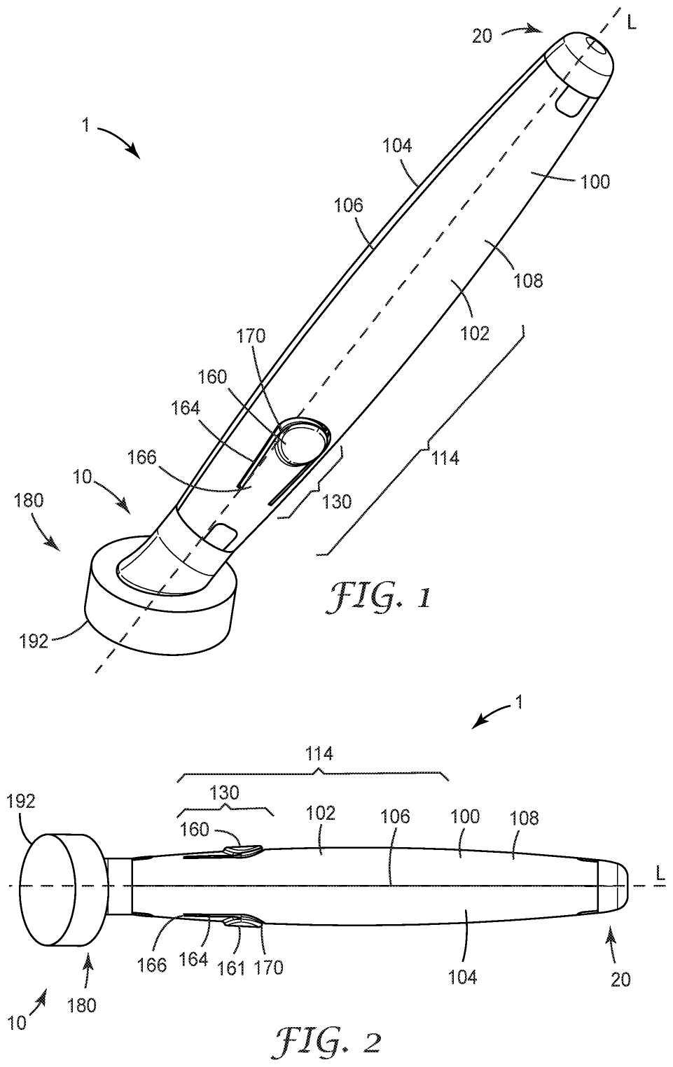

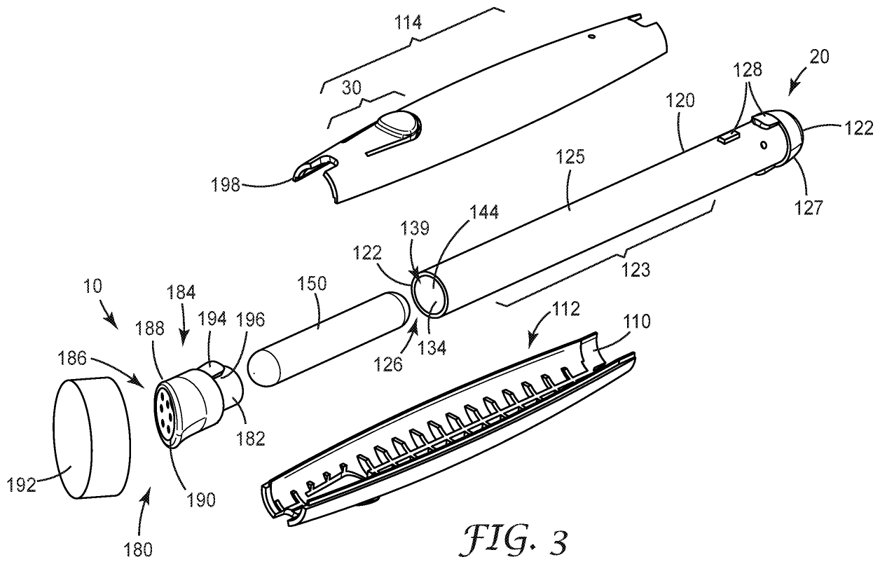

FIGS. 1-7 illustrate one embodiment of an applicator 1. The applicator 1 includes a cover 100, a deformable body 120 disposed within the cover 100, and a frangible ampoule 150 containing a solution that is disposed within the deformable body 120. The applicator 1 has a distal portion 10, a proximal portion 20, and an activation region 30 between the distal portion 10 and proximal portion 20. In this embodiment, the cover 100 extends along a central longitudinal axis L that defines a longitudinal direction.

The cover 100 may include a first half 102 and a second half 104 coupled to each other at a midline 106. The midline 106 extends parallel to the longitudinal axis L. The first and second halves 102, 104 define an exterior surface 108 of the cover 100. An interior surface 110 of the cover 100 defines a cover cavity 112 (FIGS. 3 & 4). Further, the cover 100 includes a handle section 114 that may be generally elongated and tapered along the longitudinal axis L towards the distal and proximal portions 20, 22 to facilitate gripping of the applicator 1 by the user. The activation region 30 of the applicator 1 is proximate the handle section 114 of the cover 100. The activation region 30 includes one or more pinch-point levers 160, 161 to break the frangible ampoule 150 contained within deformable body 120 when there is an application of force to the one or more pinch point levers 160, 161. The force may be applied by, for example, a user holding the applicator 1 along the palm of his/her hand at the handle section 114 and squeezing the one or more pinch point levers 160, 161 between the user's fingers and thumb. Actuation of the one or more pinch point 160, 161 levers provides additional leverage and force amplification when transferring the force from the activation region 30 to the ampoule 150.

At least one pinch point lever 160 is flexibly connected to the exterior surface 108 of the cover 100. The pinch point lever 160 includes a free section 170 and a hinge 166. The hinge 166 is flexibly connected to the exterior surface 108 of the cover 100, while the free section 170 easily actuates relative to the cover 100. For example, in one embodiment, which will be described below, the free section 170 is disconnected from the exterior surface 108 of the cover 100 by cut outs 164. In other embodiments, the free section 170 might include thinner wall portion between the free section 170 and the exterior surface 108 or an accordion-shaped or elastomeric wall portion between the free section 170 and the exterior surface 108.

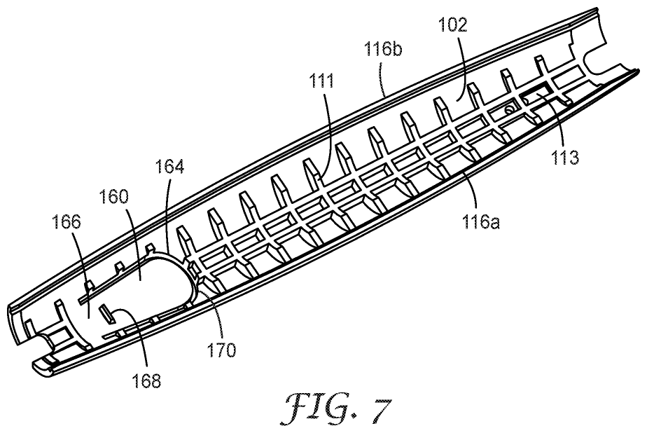

In one embodiment, the activation region 30 includes a first pinch-point lever 160 flexibly connected to the first half 102 of the cover 100, and a second pinch-point lever 161 flexibly connected to the second half 104 of the cover 100. In other embodiments, the cover 100 may include a single pinch point lever. The one or more pinch point levers 160, 161 may include a finger pad 162 engaged by a user during actuation of the pinch point levers 160, 161. The one or more pinch point levers 160, 161 may be defined by a cutout 164 formed within the cover 100. Further, the one or more pinch point levers 160, 161 may be integral with the cover 100 at a flexible hinge (i.e.: living hinge) 166. The integral configuration of the pinch point levers 160, 161 with the cover 100 prevents the levers 160, 161 from catching objects that may brush against the applicator 1 (e.g.: a users clothes). This is in contrast to the large wings or levers that are required to deform the thick walls of traditional applicators when breaking the ampoule 150. The hinge 166 is connected to the exterior surface 108 of the cover 100. Opposite the hinge 166 may be the free section 170 that is disconnected from the exterior surface 108 of the cover 100 and is depressible towards the deformable body 120. When the pinch point levers 160, 161 are engaged by a user and pressure is applied to the finger pads 162, the pinch point levers 160, 161 rotate about the hinge 166 and press downward against the deformable body 120 to deflect a flex region 128 of the deformable body 120. Deflection of the flex region 128 of the deformable body 120 crushes the ampoule 150, releasing the solution. The hinge 166 may include a stress concentrator 168 which defines a rotation point for each pinch point lever 160, 161 (FIG. 7). In other embodiments, the stress concentrator 168 may be formed on the finger pad 162 whereby the stress concentrator 168 forms a focused contact point between the levers 160, 161 and the deformable body 120. In this embodiment, the stress concentrator 168 facilitates breaking of the ampoule 150 by concentrating the break force of the levers 160, 161 on the flex region 128 of the deformable body 120.

In the illustrated embodiment, the cutout 164 forms a tear drop configuration that tapers inwards towards the distal portion 10 of the applicator 1. However, other configurations of the cutout 164 are possible, including ovular, parallelogrammatic or triangular cutouts 164 capable of forming a living hinge integrated with the applicator 1 cover 100.

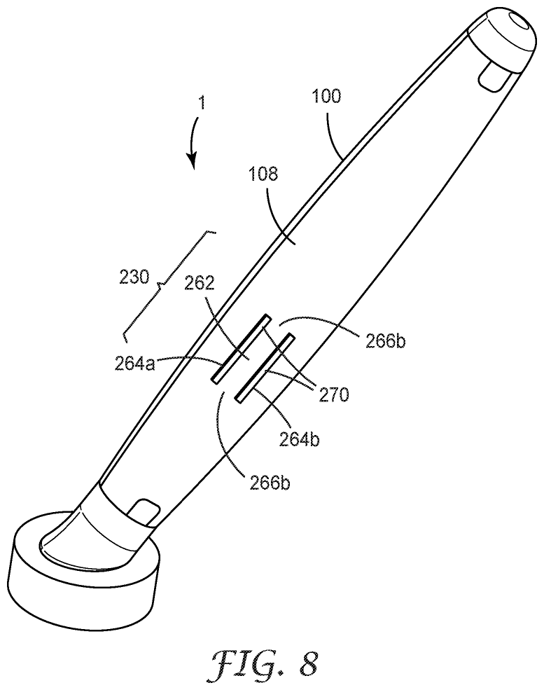

FIG. 8 illustrates a second embodiment of a pinch point lever 260 at an activation region 230. In this embodiment, the pinch point lever 60 includes a free section 270 formed by two slit cutouts 264a, 264b that define a depressible portion 262. The depressible portion 262 is connected the exterior surface 108 of the cover 100 by a first and second hinge 266a, 266b. The embodiment of FIG. 8 functions in substantially the same manner as that of the embodiment illustrated in FIGS. 1-7, in that the depressible portion 262 is pressed against the deformable body 120 to crush the ampoule 150 residing within the deformable body 120.

The shape and size of the pinch point levers according to the embodiment are only examples and other configurations are possible provided that at least one pinch point lever 160 is flexibly connected to the cover 100 to depress against the deformable body 120 to crush the ampoule 150 residing within the deformable body 120.

With reference to FIGS. 3-6, the deformable body 120 is disposed within the cover cavity 112 and is adjacent to the interior surface 110 of the cover 100. The deformable body 120 may be a cylindrical tube having a distal end 121, a proximal end 122, a midsection 123 between the distal end 121 and the proximal end 122 an interior surface 124, and an exterior surface 125. The deformable body 120 may be ovular in shape, which facilitates deflection of the deformable body by the pinch point levers 160, 161. Further, the deformable body 120 fits within cover such that there is space between the exterior surface 125 of the deformable body 120 and the interior surface 110 of the cover. Adequate separation between the cover 100 and the deformable body 120 provides the pinch point levers 160, 161 with sufficient leverage to break the ampoule 150. The deformable body 120 includes a at least one opening 126 proximate the distal end 121 that allows the solution within the frangible ampoule 150 to exit the deformable body 120.

The midsection 123 of the deformable body 120 aligns to the activation region 30 of the applicator 1. In particular, the midsection 123 aligns to the pinch point lever 160. Therefore, in embodiments like those described above where the pinch point lever 160 includes cut outs 164 to provide a flexible connection of the pinch point lever 160 with the cover 100, the deformable body 120 at the midsection 123 is a continuous material. When the midsection 123 is a continuous material and there are substantially no openings Therefore, any of the solution will not leak and exit from the applicator 1 at the cutout 164 in the cover 100 at the pinch point lever 160. Instead, any openings in the deformable body 120 are at the distal end 121.

The proximal end 124 of the deformable body 120 may be closed such that the solution does not exit at the proximal end 124. The deformable body 120 may be continuously formed to be closed at the proximal end 124 or may be closed by a rear plug 127. The rear plug 127 may include one or more retention features 128 configured to couple the deformable body 120 to the first and second halves 102, 104 of the cover 100. The retention features 128 prevent the deformable body 128 from spinning or moving within the cover 100. The rear plug 127 is preferably integral with the deformable body 120. However, the rear plug 127 may be a separate component that is inserted into the proximal end 124 of the deformable body 120.

The deformable body 120 receives the frangible ampoule 150. The frangible ampoule 150 may be composed of a breakable material such as glass and is hermetically sealed to contain a solution. The frangible ampoule 150 may be retained near an opening 126 of the deformable body 120. In some embodiments, the deformable body 120 includes a cross wall 142 or spacer (FIGS. 4 and 6) extending from the proximal end 124 of the deformable body 120 to the midsection 123. The cross wall 142 prevents the frangible ampoule 150 from moving about an interior cavity 139 of the deformable body 120. At least a portion of the frangible ampoule 150 is located in the activation region 30. When the pinch point levers 160, 161 are compressed against the deformable body 120, the deformable body 120 crushes the frangible ampoule 150, releasing the solution. It is beneficial that the deformable body 120 be both thin and flexible enough to easily deform upon pressing of the pinch point lever 160, 161, while additionally preventing shards of glass from piercing the deformable body 120. Therefore, the applicator 1 does not require thicker walls and large levers of traditional applicators that are often difficult to operate with one hand because the deformable body 120 and pinch point levers 160, 161 provide a mechanism for easily fracturing an ampoule 150 while operating the applicator 1 with one hand, and also protecting the user from shards of the fractured ampoule 150.

Prior to fracturing, the frangible ampoule 150 may desirably act as a protective barrier that encapsulates the solution contained therein from the outside environment. For example, the frangible ampoule 150 may be resistant to environmental changes encountered by the applicator 1 during manufacture, transport, and use of the applicator 1, such as, for example, the vacuum and chemicals, e.g., ethylene oxide, that the applicator 1 may be exposed to during a sterilization process, conditions that could damage or destroy the solution inside the frangible ampoule 150.

Commonly, the frangible ampoule 150 may be formed of a brittle material, such as, for example, a glass. Suitable brittle materials for forming the frangible ampoule 150 include, for example, a soda-lime glass, a borosilicate glass, an onion skin borosilicate glass, a polymer, a ceramic, and combinations thereof. Such materials are desirably brittle and will fracture when compressed. Typically, the solution contained within the frangible ampoule 150 is released by applying an external force to the applicator 1 in the activation region 30 sufficient to break open the frangible ampoule 150. In some embodiments, score lines or other features that provide local areas of weakness in the brittle material may be included to control breaking and/or reduce the force required to break the frangible ampoule 150.

The size and shape of the frangible ampoule 150 is selected to be compatible with the dimensions of the cover 100 and deformable body 120, as well as the desired volume of solution to be applied. For use in preparation for small surgical procedures, the amount of solution in the ampoule 150 should generally be sufficient to cover an area of, such as, for example about 10 square centimeters. The amount of solution in the ampoule 150 may be sufficiently more or less when attempting to cover areas of greater or smaller surface area, for instance, an area of about 5 to 20 square centimeters or an area of about 1 to 30 square centimeters. The size of the frangible ampoule 150 may be selected to be appropriate for the volume of solution to be delivered in order to minimize product costs and waste. Smaller frangible ampoules 50 may result in products containing longer shelf lives. As a nonlimiting example, it has been found that solutions containing cyanoacrylate adhesives have longer shelf lives in small vials compared to larger vials even when the vials are flushed with an inert gas before sealing. The applicator 1 can accommodate relatively smaller vials with an increased shelf life and cost effectiveness. In some embodiments, use of a frangible ampoule 150 having a thinner wall may be used, which increases the ease with which the ampoule 150 may be fractured. In some embodiments, the frangible ampoule 150 contains, for example, 0.1 mL to about 2 mL of a solution.

In some embodiments, the frangible ampoule 150 may contain an antiseptic preparation. Examples of suitable antiseptic preparations include those described in U.S. Pat. No. 4,584,192 and those described in U.S. Pat. No. 4,542,012. Other useful fluids include antiseptic preparations, such as, for example, iodophoric skin tinctures, such as DURAPREP Surgical Solution, commercially available from 3M Company, Saint Paul, Minn., USA. In some embodiments, the ampoule 150 may be filled with a composition that includes an antimicrobial agent such as iodine, an iodine complex (such as, for example, iodophors), chlorhexidine, chlorhexidine salts (such as, for example, chlorhexidine digluconate and chlorhexidine diacetate), or combinations thereof. Other exemplary antimicrobial agents include C2-C5 lower alkyl alcohols, fatty acid monoesters of glycerin and propylene glycol, polymers that include a (C12-C22) hydrophobe and a quaternary ammonium group, polyquaternary amines (such as, for example, polyhexamethylene biguanide), quaternary ammonium silanes, silver, silver salts (such as silver chloride), silver oxide and silver sulfadiazine, methyl, ethyl, propyl and butyl parabens, octenidine, peroxides (such as, for example, hydrogen peroxide and benzoyl peroxide), and the like, as well as combinations thereof. In other embodiments, the frangible ampoule 150 may contain, for example, medical preparations such as skin barrier products, adhesion enhancing products, adhesive tape trauma protectants, and pharmaceutical agents such as analgesics. As a nonlimiting example, the frangible ampoule 150 may contain a skin barrier product, such as CAVILON Advanced Skin Protectant, commercially available from 3M Company, Saint Paul, Minn., USA. In some embodiments, the contents of the frangible ampoule 150 include a cyanoacrylate monomer.

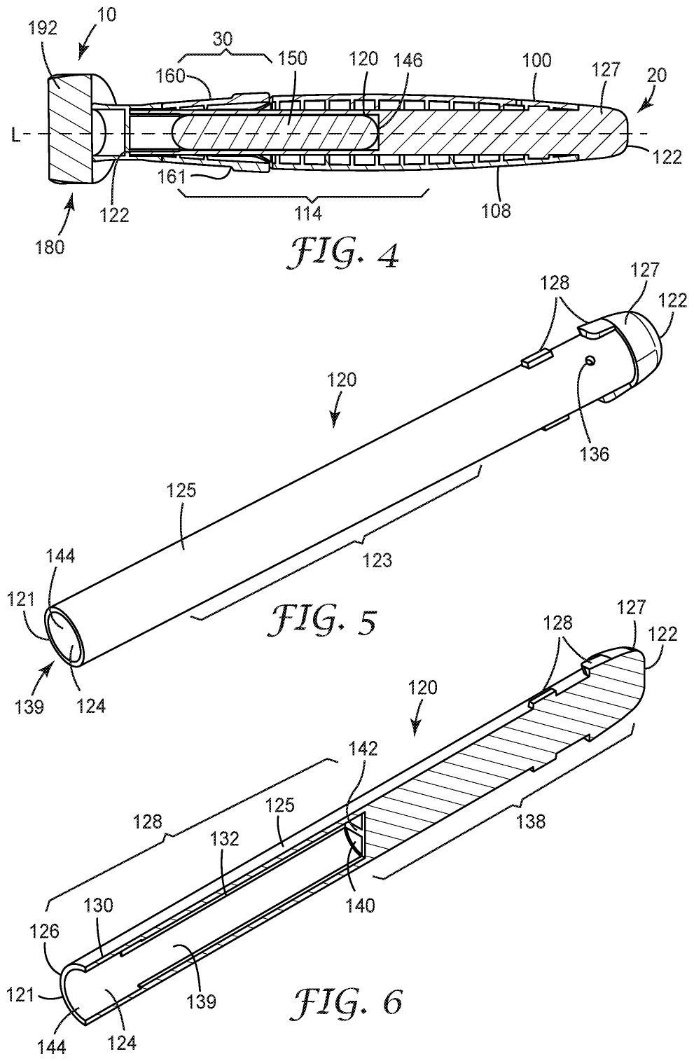

Referring to FIGS. 3-6, the deformable body 120 prevents fragments of the frangible ampoule 150 from puncturing the cover 100, and more specifically the activation region 30 wherein shards would otherwise be able to escape the cover 100. The frangible ampoule 150 is accommodated within the deformable body 120 near a thin flex region 128 that facilitates fracturing of the frangible ampoule 150 when the activation region 30 is compressed (FIG. 6). The flex region 128 of the deformable body 120 includes a thin outer wall 130 which allows reduced force to deflect the deformable body 120 when crushing the ampoule 150. The flex region 128 can include one or more raised ridges 132 that extend longitudinally along an interior surface 134 of the deformable body 120 (FIG. 6). The one or more ridges 132 engage the frangible ampoule 150 and allow space between the interior surface 134 of the deformable body 120 and the frangible ampoule 150 for flow of the solution and venting of the applicator 1, which prevents a vapor lock wherein solution is unable to flow from the applicator 1. Moreover, a deformable body vent 136 located near the proximal end 124 connects the internal air space to the outside of the applicator 1 to enable full venting of the deformable body 120 (FIG. 5).

In one embodiment, the deformable body 120 may include a support region 138 to give the deformable body 30 and applicator 1 rigid support lengthwise (FIG. 6). The support region 138 enables the applicator 1 to have a longer shape that facilitates handling of the applicator 1 by a user and includes a thick outer wall 140 defining the support region 138. The interior surface 134 of the deformable body 120 at the support region 138 includes one or more cross walls 142 that extend width-wise from one side of the thick outer wall 140 to an opposite side of the thick outer wall 140 (FIG. 6). The one or more cross-walls 142 extend lengthwise from the proximal end 124 of the deformable body 120 to the flex region 128. The one or more cross walls 142 provide flexing stiffness and strength to the deformable body 120.

Referring to FIG. 3, the opening 144 of the deformable body 120 is configured to accommodate an annular coupling surface 182 of a head region 180 in order to couple an end cap 184 to the distal end 122 of the deformable body 120. The annular coupling surface 146 may be inserted into the opening 144 of the deformable body 120 and may be sealed by ultrasonic welding, spin welding, solvent bonding, hot plate welding, or other known plastic attachment techniques capable of forming a leak tight seal. A mouth 186 of the end cap 184 is defined by a radiused tip 188 that protrudes from the end cap 184. Further, the mouth 186 includes a plurality of apertures 190 configured to allow the solution to pass through the mouth 186 of the end cap 184 and out of the applicator 1. The small size of the apertures 190 prevents shards from the crushed ampoule 150 from exiting the applicator 1 along with the solution, preventing the shards from entering an absorbent component 192 and harming the patient on whom the applicator 1 is being used.

Additionally, the end cap 184 has one or more locating extensions 194 extending rearwardly from the tip 188 of the cap. An annular gap 196 defines a space between the extensions 194 and the coupling surface 812, wherein the gap 196 is configured to accommodate the distal end 122 of the deformable body 120. The extensions 196 are configured to fit into one or more mating grooves 198 located near the distal portion 10 of the applicator 1 cover 100. The coupling of the extensions 194 and the mating grooves 198 align the end cap 184 and the cover 100, securing the end cap 184 in place. Thus, the end cap 184 is prevented from rotating or spinning around the longitudinal axis L of the applicator 1.

The absorbent component 192 may be attached to the head region 180 wherein the absorbent component 192 is coupled to the end cap 184 near the distal portion 10 of the applicator 1. The absorbent component 192 may be attach around the radiused tip 188 of the end cap 184 (FIG. 3). In some embodiments, the absorbent component 192 may be attached at the head region 180 of the applicator 1 by, for example, hot plate welding. The absorbent component 192 may be attached to the head region 180 by heating end cap 184 to a suitable temperature and then bringing the heated end cap 184 and the absorbent component 192 into contact under pressure. In other embodiments, the absorbent component 192 may be attached to the head region 180 by other means, such as, for example an adhesive, a spin weld, an ultrasonic weld, or combinations thereof.

The absorbent component 192 may, among other functions, aid in control of the flow rate and distribution of the contents of a frangible ampoule 150 after the contents have been released from the frangible ampoule 150. The absorbent component 192 may be formed of any suitable porous solution such as, for example, a sponge, a woven material, a nonwoven material, a screen, a mesh, and combinations thereof. Materials suitable for use as the absorbent component 192 may include, for example, polyester polyurethane and polyester polyether open-cell foams.

Referring to FIGS. 5-7, The first and second half 102, 104 of the cover 100 are substantially symmetrical. However, the first half 102 of the cover 100 may include a lip 116a, while the second half 104 may contain a groove 116b (FIG. 7). The groove 116b is configured to accommodate the lip 116a to couple the first half 102 to the second half 104. The first and second halves 102, 104 may be sealed by ultrasonic welding, spin welding, solvent bonding, hot plate welding, or other known plastic attachment techniques.

In order to avoid creating a vacuum and restricting the flow through the applicator 1, a means of maintaining atmospheric pressure in the device is preferably employed. The cutouts of the pinch point levers, in conjunction with the deformable body vents 118, connects the outside atmosphere to the interior of the applicator 1. The interior surface 110 of the cover 100 includes a plurality of ribbed features 111 to give the cover 100 strength (FIG. 7). The underside of the cover 100 includes mating holes 113 configured to mate with alignment notches 125 that extend from the exterior surface 135 of the deformable body 120 (FIGS. 3, 5 and 6). The notches 125 couple the deformable body 120 to the cover 100 and prevent spinning of the deformable body 120 within the cover 100.

Applicator 1 covers may be molded from polymers such as, for example, polyethylenes, linear low density polyethylenes, medium density polyethylenes, high density polyethylenes, branched polyethylenes, polypropylenes, and combinations thereof. Applicator 1 bodies of the present disclosure were molded from a combination of linear low-density polyethylene and medium density polyethylene and used to make applicator is of the present disclosure. The deformable body 120 may be composed of a polymer such as low density polyethylene, medium density polyethylene, or high density polyethylene.

Although specific embodiments have been shown and described herein, it is understood that these embodiments are merely illustrative of the many possible specific arrangements that can be devised in application of the principles of the invention. Numerous and varied other arrangements can be devised in accordance with these principles by those of skill in the art without departing from the spirit and scope of the invention. The scope of the present invention should not be limited to the structures described in this application, but only by the structures described by the language of the claims and the equivalents of those structures.

What is claimed is:

1. An applicator for applying a solution to a surface, the applicator comprising:
a cover comprising an exterior surface and an activation region;
a deformable body disposed within the cover at the activation region;
a frangible ampoule disposed within the deformable body, wherein the frangible ampoule contains the solution;
a pinch point lever disposed within the activation region of the cover, wherein the pinch point lever is flexibly connected to the exterior surface of the cover; and
wherein actuation of the pinch point lever against the deformable body breaks the frangible ampoule.

2. The applicator of claim 1, wherein the cover comprises a cover interior surface surrounding a cover cavity.

3. The applicator of claim 1, wherein the cover comprises a distal portion and a proximal portion, wherein the distal portion comprises an opening for releasing the solution from the applicator.

4. The applicator of claim 1, wherein the deformable body is adjacent to the interior surface of the cover.

5. The applicator of claim 1, wherein the deformable body has a proximal end and a distal end and a midsection, wherein the deformable body has a continuous wall extending circumferentially around the midsection.

6. The applicator of claim 1, wherein the deformable body has an opening at the distal end.

7. The applicator of claim 1, wherein the deformable body comprises a support region including one or more cross walls that extend longitudinally across the support region.

8. The applicator of claim 1, wherein the deformable body comprises one or more raised ridges extending from the interior surface of the deformable body to provide air space between the deformable by and cover.

9. The applicator of claim 1, wherein the deformable body further comprises a flex region, an interior surface and one or more ridges extending along the interior surface at the flex region.

10. The applicator of claim 1, wherein the pinch point lever includes a hinge portion that is integral to the cover.

11. The applicator of claim 1, wherein the pinch point lever has a free section and a hinge end, wherein the free section is an end of the pinch point lever opposite the hinge end.

12. The applicator of claim 11, wherein the hinge end is connected to the exterior surface of the cover and the free end is disconnected from the exterior surface of the cover.

13. The applicator of claim 1, wherein the pinch point lever has a first hinge end and a second hinge end and a free section between the first hinge end and the second hinge end.

14. The applicator of claim 1, further comprising a second pinch point lever disposed within the activation region of the cover, wherein the second pinch point lever is flexibly connected to the exterior surface of the cover.

15. The applicator of claim 1, further comprising a head region located at the distal portion of the cover, wherein the head region comprises an aperture.

16. The applicator of claim 1, further comprising an absorbent element at the distal portion of the cover.

17. The applicator of claim 1, wherein the ampoule is hermetically sealed.

18. A method of applying a solution to a surface, the method comprising:
providing the applicator of any one of the preceding claims;
pressing the pinch point lever against the deformable body;
breaking the frangible ampoule such that the solution releases through the deformable body and the cover.
